(19) United States
(12) Patent Application Publication
(10) Pub. No.: US 2006/0104856 A1
(43) Pub. Date: May 18, 2006

(54) Sterilization wrap with fastening means

(75) Inventors: Melissa Robyn Farrell, Woodstock, GA (US); Tara Denise Smith, Marietta, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/992,315

(22) Filed: Nov. 18, 2004

(51) Int. Cl. A61L 2/00

(52) U.S. Cl. 422/1; 422/28; 422/34; 422/292

(57) ABSTRACT

Disclosed herein is a sterilization wrap system suitable for use, as an example, in wrapping surgical instruments and supplies for sterilization, transportation and storage. The sterilization wrap system includes an adhesive on the surface which activates upon exposure to sterilization conditions to hold the sterilization wrap system in a closed configuration about an item to be sterilized.

### BACKGROUND

Personnel in the Central Service Room (CSR) or the Sterile Processing Department (SPD) of hospitals are commonly charged with the responsibility of packaging surgical supplies to ensure that the sterility of the packaged contents is maintained from sterilization to the point of reuse. Several activities are involved in the task of sterile supply delivery to the operating room and other units.

Much of the surgical instruments and supplies used in the operating room are reusable. These supplies typically include such things as clamps, scalpel blade handles, retractors, forceps, scissors, surgeon's towels, basins and the like. All of these supplies must be collected after each procedure, placed in a sterilization packaging system and sterilized before they can be used again in another procedure. The sterilization packaging systems used must be of the size and shape to accommodate the items to be sterilized and must be compatible with and withstand the physical conditions of the sterilization process.

Common sterilization packaging systems include sealable pouches and sterilization wraps. Generally, sterilization pouches are used for small, lightweight objects. A sterilization pouch is made of flexible materials which are formed in a pouch having an open end, into which the instrument to be sterilized is place. The open end is typically sealed closed with an adhesive strip or a peelable heat seal. Examples of such sterilization pouches can be found in U.S. Pat. No. 5,459,978 to Weiss et al. and in U.S. Pat. No. 3,991,881 to Augurt.

Alternatively, sterilization wrap is generally used for the sterilization of larger, heavier and/or irregularly shaped objects. In particular, sterilization wrap is used to wrap sterilization trays containing several objects; often the tray will contain all of the instruments needed for a single particular medical procedure and can weigh between 5 to 30 pounds. Generally, metal supplies are placed in stainless steel sterilization trays, while soft goods such as surgeon's towels, drapes, and gowns are wrapped directly. The sterilization wrap is usually a woven or nonwoven material which when wrapped around the tray or package contents, the tray or package contents are fully enclosed within the folds of the wrap. Wrapping in a certain prescribed manner will permit the entry of sterilizing vapor/gas or other medium to sterilize the contents of the tray while denying the ingress of contaminants such as bacteria and other infectious causing materials or their vehicles after sterilization.

In order to promote and maintain the sterility of the packaged contents, the Association of Operating Room Nurses (AORN) has developed certain recommended practices for the wrapping and handling of in-hospital processed packages. It is common practice among many hospitals as recommended by the AORN to “double wrap” in-hospital processed packages with two layers of barrier material. This minimizes the probability of a breach due to a flaw in any one layer of material.

A primary method of double wrapping is “sequential” in nature in that the package contents are first wrapped by one sheet of sterilization wrap and then wrapped again by another sheet of sterilization wrap. Another method of double wrapping is “simultaneous” in nature in that the package contents are wrapped by two sheets of sterilization wrap at the same time. That is, two sheets of sterilization wrap are aligned one on top of the other, and the item to be wrapped is placed on top of the two sheets, then the item is wrapped by both sheets of material at the same time. Products have been developed that reduce the labor required in simultaneous wrapping by joining an outer and inner layer such that the layers can be manipulated as a unitary laminate wrapper. For example, one such product is KIMGUARD® ONE-STEP® produced by Kimberly-Clark Corporation which is generally described, for example, in U.S. Pat. No. 5,635,134 and 5,688,476. Other such two-ply sterilization wraps can be found U.S. Pat. No. 6,406,764 to Bayer and U.S. Pat. No. 6,517,916 to Bayer et al.

Common means of sterilizing instruments include, among others, autoclaving with steam, exposure to ethylene oxide gas, and exposure to hydrogen peroxide plasma, as is done with the STERRAD® Sterilization System from Advanced Sterilization Products, Irvine, Calif. Once the wrapped tray and its contents have been sterilized, the wrapped tray is typically stored until it is needed for a surgical procedure.

Once needed, the wrapped tray is transported to the point of use, typically an operating room. During storage and transfer to the operating room, the wrapped tray may be handled several different times. Each time the wrapped package is handled, there is a potential that the sterile nature of the package contents can be compromised. The two most common ways the wrapped package can be compromised are a tear or other breach of the wrapping material, and wetness or foreign materials identified on the outer wrapper. Either of which would warrant re-processing of the tray and contents.

Studies have been used to track packages from initial wrapping, all the way through sterilization, storage, handling, transfer, unwrapping and ultimate reuse. These studies indicate that the frequency of compromising wrapped items due to tears or holes has been declining because of improved handling and storage techniques and practices, as well as improved sterilization packaging products and materials. One of the main thrusts behind such efforts has been economics. Every time a sterile package is compromised, it must be taken out of circulation, unwrapped, rewrapped, and resterilized with a new sterilization wrapper before it can properly be reused. This wastes time and money.

The method of wrapping helps keep the sterilization package sealed. Selecting an adequately sized sterilization wrap having good drapeability and making crisp folds and tucks, according to the wrapping practice used, helps keep the sterilization wrap folded about the items to be sterilized. Additionally, the loose ends of the sterilization wrap that remain after making the final fold are secured with a piece of adhesive tape. In addition to holding the loose ends of the sterilization wrap, the adhesive tape is often designed to change color upon being exposed to sterilization conditions. Such color changing tape acts as an external indicator as to whether or not a sterilization package has been subjected to sterilization conditions.

However, such a tape is an additional material that needs to be available when preparing sterilization packages. Also, the tape holds the sterilization package closed by merely holding down the loose end of the sterilization wrap. The sterilization package is held closed by the combination of the quality of the folding and the bond the adhesive tape can form where it contacts the sterilization wrap material. As such, the sterilization package is susceptible of becoming unwrapped if the tape is dislodged in pre- or post-sterilization handling.

Consequently, there is a need for a new sterilization wrap system that reduces the likelihood of re-processing.

### SUMMARY OF THE INVENTION

The present invention is directed to a sterilization wrap system having at least a first sheet with an adhesive present on that first sheet which is activated by sterilization conditions. Such sterilization conditions may be steam, ethylene oxide, or hydrogen peroxide plasma sterilization conditions. The present invention includes an embodiment where the adhesive capable of activation is present on a surface of a sterilization wrap system made of two sheets joined together. The sheets of the sterilization wrap system can be spunbond/meltblown/spunbond nonwoven web laminates, and in one embodiment, can contain polypropylene.

In one embodiment, the adhesive can be placed in at least one discrete area of the surface of the sterilization wrap system such that that total surface area of the adhesive is less than that of the surface of the sterilization wrap system. In another embodiment, the sterilization wrap system can also include a secondary fastening means.

The adhesive can additionally indicate that it has been activated. The adhesive may indicate that is has been activated by changing colors upon activation.

The invention also includes a wrapped package formed by the combination of a sterilization wrap system, such as discussed above, and an article to be sterilized. The article to be sterilized, in one embodiment, is at least one reusable medical instrument.

The invention also provides a method for sterilizing and article which includes the steps of providing an article to be sterilized, wrapping the article with a sterilization wrap system and exposing the wrapped article to sterilizing conditions for a sufficient time to sterilize the article. The sterilization wrap system used in the method is a sterilization wrap system of two sheets of spunbond/meltblown/spunbond nonwoven laminate joined together and an adhesive present on the surface of the sterilization wrap system which is activated under sterilization conditions. In one embodiment of this method the sterilization conditions may be steam sterilization conditions, ethylene oxide sterilization conditions, or may be hydrogen peroxide plasma sterilization conditions. In another embodiment of this method, after the article is wrapped and prior to the exposure to sterilization conditions, the sterilization wrap system is secured in a closed configuration with a secondary fastening means.

### BRIEF DESCRIPTION OF THE DRAWINGS

FIG. 1 is a perspective view of a sterilization wrap system according to the present invention with a sterilization tray ready for wrapping placed on top of the sterilization wrap system.

FIG. 2 is a cross-sectional side view of the embodiment of a sterilization wrap system illustrated in FIG. 1.

FIG. 3 is a perspective view of a multiple ply, single step sterilization wrap system according to the present invention with a sterilization tray ready for wrapping placed on top of the sterilization wrap system.

FIG. 4 is a cross-sectional side view of the embodiment of a multiple ply, single step sterilization wrap system illustrated in FIG. 3.

FIGS. 5 through 8 are bottom plan views of sterilization wrap systems according to the present invention with alternate placement of sealing adhesive.

FIGS. 9(A) through 9(D) sequentially illustrate a method for wrapping a sterilization tray using a sterilization wrap system according to the present invention.

FIGS. 10 and 11 are bottom plan views of sterilization wrap systems according to the present invention.

### DETAILED DESCRIPTION

Disclosed herein is a sterilization wrap system suitable for use with either sequential or simultaneous wrapping procedures for wrapping, sterilizing, storing and using sterilized items such as surgical supplies. While the present invention will be described in conjunction with its use in hospital and surgical room procedures, the sterilization wrap system of the present invention is intended for use wherever there is a need for sterilized materials. Consequently, the following description of the present invention should not be considered a limitation as to the scope of use of the present invention.

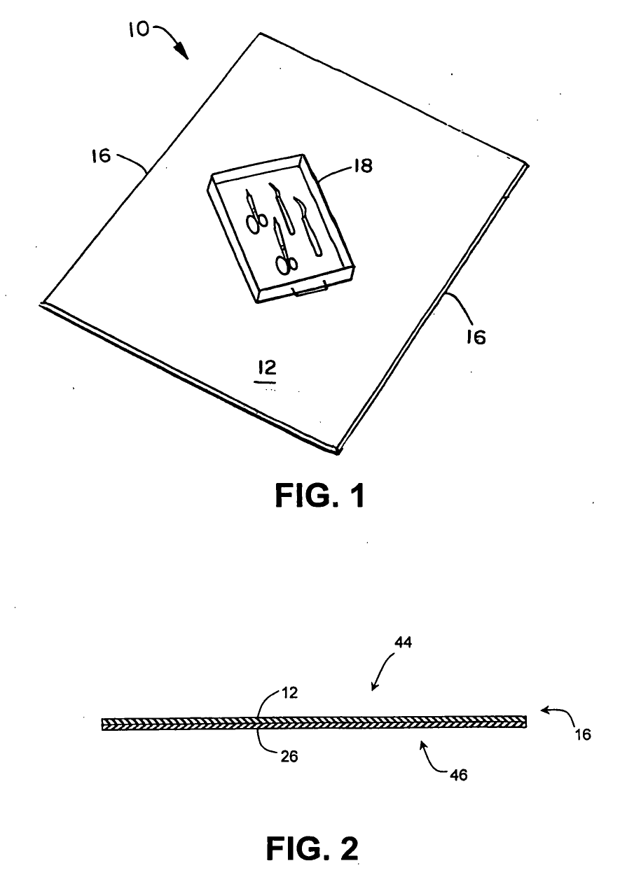

The sterilization wrap of the present invention can embody a single sheet of material to which a sealing adhesive capable of activating upon exposure to sterilization conditions has been applied. As illustrated in FIGS. 1 and 2, the sterilization wrap system 10 is made of a first sheet 12 of material upon which such a sealing adhesive 26 has been applied to one entire surface of the first sheet 12. The sterilization wrap system 10 has a first exterior surface 44 comprising the upper surface of the first sheet 12 which, in this illustration of FIGS. 1 and 2, is free of any sealing adhesive 26. The sealing adhesive 26 is present on the opposite side of the first sheet 12 and makes up the second exterior surface 46 of the sterilization wrap system 10.

To wrap an item, such as a sterilization tray 18 as shown in FIG. 1, the item is placed on top of the sterilization wrap system 10 in contact with the first exterior surface 44 such that the four corners of the sterilization wrap system 10 can be folded over the item one at a time to fully wrap the item, to form a wrapped package. During and after this wrapping, the sterilization tray 18 would only come into contact with the first exterior surface 44 of the sterilization wrap system 10. The resulting exterior surfaces of the wrapped item would be the second exterior surface 46 made up of the sealing adhesive 26. When following a sequential wrapping procedure, an additional sheet of the same sterilization wrap system 10 as shown in FIG. 1 can wrapped about the wrapped item in the same manner to form a finished wrapped package having sealing adhesive 26 present on the exposed exterior surfaces.

The sterilization wrap system must be of a size large enough to fully wrap the items to be sterilized. Each fold of the sterilization wrap system 10 must fold over most of the item to be sterilized, each subsequent fold overlapping the previous fold, leaving the item to be sterilized completely encompassed within the folds of the sterilization wrap system. Generally, the sterilization wrap systems come in several sizes to wrap various size items and trays. Typical sizes include 18, 24, 30, 36, 40, 45, 48 and 54 inch square sheets as well as 54 inch by 72 inch rectangular sheets (1 inch=2.54 cm).

Once properly wrapped, the finished wrapped package can then be transferred to the sterilizing equipment and exposed to sterilization conditions. As mentioned above, such sterilization conditions can include steam, ethylene oxide, or hydrogen peroxide plasma sterilization conditions. The sealing adhesive 26 is to be formulated so that when exposed to such conditions the sealing adhesive 26 is activated to form or strengthen bonds that keep the wrapped package in a completely wrapped configuration, also known as a “closed” configuration. This closed configuration denies ingress of contaminants due to both the barrier properties of the sterilization wrap system materials and the tortuous path created by the folds of the sterilization wrap system.

The placement of the sealing adhesive 26 may also allow the sterilization wrap system to become completely sealed upon exposure to sterilization conditions. If the sealing adhesive 26 covers an entire surface of the sterilization wrap system, or is otherwise strategically placed, the sealing adhesive 26 may adhere the folds of the sterilization wrap system to themselves and completely seal off the tortuous path from ingress of any contaminants.

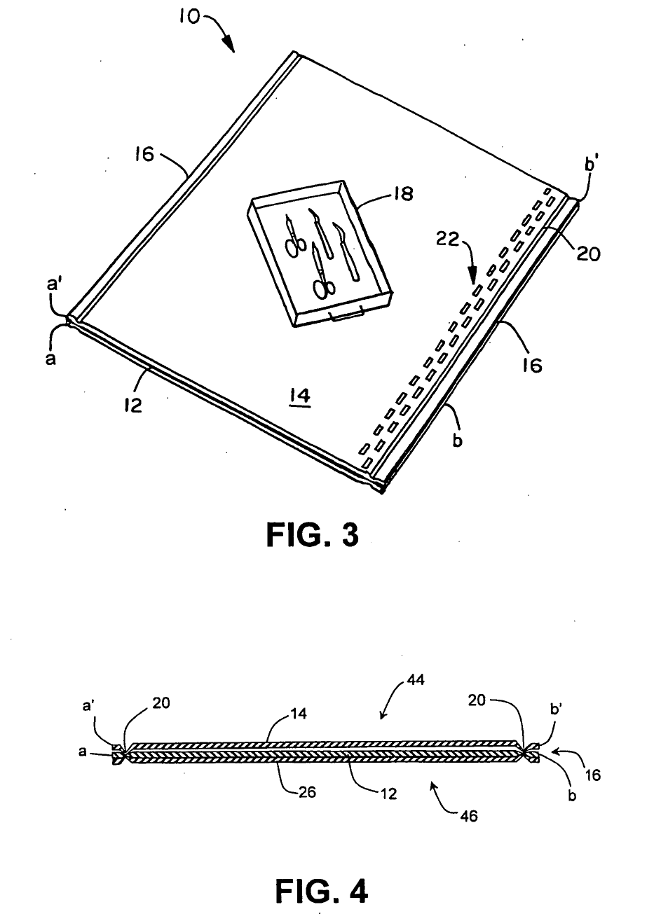

Another embodiment of the present invention includes the presence of a sealing adhesive 26, on a multiple-ply sterilization wrap system, as shown in FIGS. 3 and 4. Along with a first sheet 12, as in the previous embodiment, this sterilization wrap system also includes a second sheet 14. As in the single sheet embodiment, the multiple-ply embodiment is shown in FIG. 4 with the sealing adhesive 26 on one entire surface of the first sheet 12 and makes up the second exterior surface 46. However, as shown in FIG. 3, the upper surface of the second sheet 14 makes up the first exterior surface 44, upon which the sterilization tray rests.

Such a sterilization wrap system 10, as shown in FIG. 3 can be used for simultaneous wrap procedures where both the first and second sheets 12, 14 are together wrapped about the sterilization tray 18 in a manner similar to the manner described above for wrapping the sterilization tray 18 with a single sheet. As before, the sterilization tray 18 would only come into contact with the first exterior surface 44 of the sterilization wrap system 10. The resulting exterior surfaces of the wrapped item would be the second exterior surface 46 made up of the sealing adhesive 26. The sealing adhesive 26 is to be formulated so that when exposed to sterilization conditions the sealing adhesive 26 is activated to form or strengthen bonds that keep the wrapped package in a closed configuration.

To facilitate wrapping of an item 18 such as is shown in FIG. 3, the first sheet 12, and second sheet 14 are attached to one another in a manner so as to hold the two sheets together while still maintaining their visual distinctiveness so that the end user can visually see that the item is being wrapped by multiple separate sheets of sterilization wrap. Generally the sheets will be joined about all or a portion of their peripheries 16. As specifically shown in FIGS. 3 and 4 the two sheets are joined to one another along the entire length of two generally parallel edges of the wrap, a-a′ and b-b′. The edges can be joined to one another by any number of suitable means including, but not limited to, adhesives, stitching, thermal bonding and ultrasonic bonding collectively referred to as joining. As shown in FIGS. 3 and 4 the bond sites 20 are perfected by ultrasonic bonding, are continuous, and run the entire length of the edges just interior to or along the periphery 16 on opposed sides of the first sheet 12 and the second sheet 14.

In addition to or as an alternate to the continuous bonds or seams 20, a second set of bonds 22 may be used to secure the sheets together. The second set of bonds 22 in FIG. 3 are a series of spaced-apart and separate bond points in the form of two rows of parallel but spaced apart rectangles or other shapes with the rectangles in one row being offset from the other row so that they are in overlapping relationship if the sterilization wrap system 10 were viewed edge on. This bond pattern has been used to seam sleeves on disposable surgical gowns manufactured by the assignee of record, Kimberly-Clark Corporation of Neenah, Wis. The second set of bonds 22 can be just interior of the continuous bonds 20 and serve to further join the first sheet 12 and second sheet 14 together when used alone or in conjunction with the continuous bonds 20.

It is also conceivable that joining is present on the entire periphery of the sheets, holding the sheets together at all four edges of the periphery.

The first sheet 12 of the single sheet embodiment as shown in FIGS. 1 and 2, as well as the first sheet 12 and the second sheet 14 of the multiple-ply embodiment shown in FIGS. 3 and 4, can be made from a number of materials. The sheets of sterilization wrap systems are generally characterized as falling into two main classes, reusables and disposables. Reusables are materials which, as the name suggests, can be reused, typically by washing or some other form of cleaning. Disposables, on the other hand, are usually one-use items which are discarded or recycled after their initial use. Generally, cloth, linen or other woven materials fall into the reusable category while disposables normally include nonwoven materials made from either or both natural and synthetic fibers such as paper, fibrous polymeric nonwovens as well as films which are capable of passing sterilants and retarding transmission of bacteria and other contaminants.

Nonwoven sterilization wraps have become particularly well-liked due to their barrier properties, economics and consistent quality. The nonwoven materials can be made from a variety of processes including, but not limited to, air laying processes, wet laid processes, hydroentangling processes, spunbonding, meltblowing, staple fiber carding and bonding, and solution spinning. The fibers themselves can be made from a variety of both natural and synthetic materials including, but not limited to, cellulose, rayon, nylon, polyesters, polyolefins and many other materials. The fibers may be relatively short, staple length fibers, typically less than 3 inches, or longer and substantially more continuous fibers such as are produced by spunbonding and meltblowing processes. Whatever materials are chosen, the resultant wrap must be compatible with the particular sterilization technique being used and must also provide both strength and barrier properties to maintain the sterile nature of the wrapped contents until use.

It has been found that polyolefin-based fibers and their resultant nonwovens are particularly well-suited for the production of sterilization wrap. Polypropylene spunbonded nonwovens such as are produced by the Assignee of record, Kimberly-Clark Corporation, can be used to impart strength characteristics to the sterilization wrap and in particular, the first sheet 12. In more refined embodiments, the first sheet 12 can be made from laminates such as a laminate of spunbonded and meltblown or spunbonded, meltblown, spunbonded to impart both strength and barrier properties to the first sheet 12.

A spunbonded-meltblown-spunbonded material is made from three separate layers which are laminated to one another. The method of making these layers is known and described in commonly assigned U.S. Pat. No. 4,041,203 to Brock et al which is incorporated herein in its entirety by reference. The material of Brock et al is a three layer laminate of spunbonded-meltblown-spunbonded layers which is also commonly referred to by the acronym “SMS”. The two outer layers of SMS are a spunbonded material made from extruded polyolefin fibers, or filaments, laid down in a random pattern and then bonded to one another. The inner layer is a meltblown layer also made from extruded polyolefin fibers generally of a smaller diameter than the fibers in the spunbonded layers. As a result, the meltblown layer provides increased barrier properties due to it fine fiber structure which permits the sterilizing agent to pass through the fabric while preventing passage of bacteria and other contaminants. Conversely, the two outer spunbonded layers provide a greater portion of the strength factor in the overall laminate. The laminate may be prepared using an intermittent bond pattern that is preferably employed with the pattern being substantially regularly repeating over the surface of the laminate. The pattern is selected such that the bonds may occupy about 5-50% of the surface area of the laminate. Desirably, the bonds may occupy about 10-30% of the surface area of the laminate.

A particular feature of the present invention is the specific tailoring available for each of the layers in the respective first sheet 12 and second sheet 14. While the two sheets can be identical to one another, in more refined embodiments of the present invention the first sheet 12 is designed to have higher strength properties than the second sheet 14. This is to provide a stronger barrier to tears and other possible breaches of the wrapped item from exterior objects. Conversely, in more refined embodiments of the present invention, the second sheet 14 is designed to have higher barrier properties than the first sheet 12. Adjusting the barrier and strength properties can generally be accomplished by adjusting the basis weights of the outer and inner sheets as well as the basis weights of each of the individual layers within each of the sheets. Suitable basis weight ranges for either of the sheets range between about 0.5 and about 3.5 ounces per square yard (osy) (17 to about 119 grams per square meter (gsm)).

The sealing adhesive 26 present in the sterilization wrap system of the present invention is to be formulated to activate upon exposure to sterilization conditions. Additionally, such an adhesive would need to be compatible with the materials used as the sheet(s) of the sterilization wrap system.

As used herein, the term “adhesive” refers to any substance that is adapted to bond at least portions of one or more layers or plies of sterilization wrap together by surface attachment. Such substances may be organic, inorganic, natural, synthetic or combinations thereof. Exemplary adhesives may be based on caseins, starches, gums, mucilages, terpene resins (rosin), pitches, rubbers, celluloses, rubber latexes, rubber solvents, waxes, thermoplastic resins, thermosetting resins, silicone polymers or the like.

As used herein, the terms “activation”, “activate”, “activates”, and “activated” refer to the ability of the sealing adhesive to form a bond or strengthen a bond upon the occurrence of a sterilization event. Specifically, the “sterilization event” of interest in the present invention is the exposure to sterilization conditions or exposure to sterilization conditions and subsequent removal from such conditions. The activated adhesive could form a bond with another surface or substrate. For example, the activated adhesive may form a bond with another surface coated with more of the same activated adhesive. Alternatively, a bond may be formed between the activated adhesive and the material that makes up the sheets of the sterilization system. The activated adhesive may form a bond with a particular surface or surface coating that has been selectively placed on the sterilization wrap system to hold the system in a closed configuration and/or to provide a substantially sealed interface between the sheets of the system. It is possible that activation may involve one or more of these possibilities as long as the activated adhesive would form a bond, or strengthen a bond previously made, upon exposure to sterilization conditions or exposure to sterilization conditions and subsequent removal from such conditions.

The term “sterilization conditions”, as used herein, are the conditions present during the particular sterilization methodology utilized that substantially or completely destroy bacteria and other infectious organisms in an industrial or medical product. For example, when using steam sterilization, the adhesive could be formulated to activate upon exposure to steam and/or moisture generated by condensate. Likewise, if the sterilization wrap system was for use with ethylene oxide sterilization, the sealing adhesive could be formulated to activate upon exposure to ethylene oxide. For example, such an adhesive could have alcohol or amine moieties and undergo an addition polymerization reaction when exposed to ethylene oxide.

If the sterilization wrap system was for use with hydrogen peroxide plasma sterilization, the sealing adhesive could be formulated to activate upon exposure to hydrogen peroxide. It is also possible that the sterilization conditions may also, or may alternately include other chemistries or parameters (temperature, humidity, etc.) of the particular sterilization methodology. In such cases, the adhesive could be formulated to be activated by the combination of the primary sterilants and any or all of these additional chemistries or parameters.

Adhesives that activate upon exposure to external conditions, chemicals, temperatures or irradiation have been used in other contexts. Such adhesives can be modified to meet the particular needs of the execution. One non-limiting example of such activatable adhesives is the moisture-activated, starch-based or starch-coated adhesives have been used for over a century for closing envelopes and adhering postage stamps. The moisture-activated adhesive is not sticky until moisture is applied to the adhesive surface (i.e., the envelope or stamp is licked or otherwise moistened). Exemplary discussion of such starch-based and other moisture-activated adhesives can be found in U.S. Pat. No. 25,590 to Stetson; U.S. Pat. No. 2,272,516 to Edson; U.S. Pat. No. 2,282,364 to Kunze et al.; U.S. Pat. No. 2,365,020 to Stillwell; U.S. Pat. No. 3,071,485 to Wurzburg et al.; U.S. Pat. No. 3,271,228 to Ives; U.S. Pat. No. 3,322,703 to Lindemann; U.S. Pat. No. 4,105,824 to Monte; U.S. Pat. No. 4,181,557 to Doggett et al.; U.S. Pat. No. 4,288,493 to Kropp; U.S. Pat. No. 4,325,851 to Colon et al.; and U.S. Pat. No. 5,413,829 to Brown et al.

Other adhesives have been developed for thermal activation in other contexts. Similar adhesives could be developed that are compatible to the materials of the present invention and the thermal conditions of a particular sterilization methodology used. In another non-limiting example, the adhesives used in some absorbent products and bandages have been formulated to activate at the temperature of human skin for the adhesion of the product to the skin of the wearer. For example, see U.S. Pat. No. 5,156,911 and 5,387,450 both to Stewart; U.S. Pat. No. 5,648,167 to Peck; and U.S. Pat. No. 6,565,549 to Peck. Exemplary discussion of other thermally activated adhesives can found in U.S. Pat. No. 2,223,575 to Pitman; U.S. Pat. No. 2,608,542 to Smith et al.; U.S. Pat. No. 2,608,543 to Wiswell et al.; U.S. Pat. No. 2,653,880 to Hendricks et al; U.S. Pat. No. 3,104,979 to Lawton et al.; U.S. Pat. No. 3,619,270 to Tesch; U.S. Pat. No. 3,625,787 to Radl; U.S. Pat. No. 4,135,033 to Lawton; U.S. Pat. No. 5,695,376 to Datta et al.; U.S. Pat. No. 5,905,099 to Everaerts et al.; U.S. Pat. No. 6,500,536 to Yamada et al.; U.S. Pat. No. 6,632,498 to Zimmermann et al.; U.S. Pat. No. 6,696,150 to Ikeda et al.; and U.S. Pat. No. 6,753,379 to Kawate et al.

Other non-limiting examples of adhesives that are activated by other means are also known for use in other contexts. For examples of adhesives activated by UV radiation, light or plasma gas, see U.S. Pat. No. 4,900,388 to Wyslotsky; U.S. Pat. No. 5,702,771, 6,326,450, and 6,492,019 all to Shipston et al.; U.S. Pat. No. 5,728,787 to Cantor; and U.S. Pat. No. 6,676,795 to Levandoski. An example of a microwave-activatable hot-melt adhesive is given in U.S. Pat. No. 4,906,497 to Hellmann et al. An example of use of activation by a beam of high-energy electrons is given in U.S. Pat. No. 4,803,104 to Peigneur et al. The PCT International Application WO 92/11295 to Audett et al. discusses various pressure sensitive adhesive polymers that cross-link upon activation by exposure to UV radiation, electron beam, gamma radiation, visible wavelength radiation, and microwaves.

It may be possible to formulate a sealing adhesive that would be capable of activating under more than just one type of sterilization conditions. Such a sealing adhesive would be formulated to activate upon exposure to any combination of steam, ethylene oxide or hydrogen peroxide sterilization conditions. A sealing adhesive may be formulated to activate upon exposure to UV wavelength radiation, gamma radiation, electron beam, plasma or IR sterilization conditions. It should also be apparent to one skilled in the art that future sealing adhesives could be formulated to activate under any new sterilization conditions that exist, or may be developed, to sterilize items.

Additional functionality may be formulated into the sealing adhesive. The sealing adhesive may be formulated to change in color upon sterilization. This would provide the user of such a material to visually determine from observing the color of the sealing adhesive whether or not the sealing adhesive, and thus the sterilization wrap system, had been exposed to sterilization conditions.

Another possible functionality would be the ability of the sealing adhesive to release in a particular fashion after it has been sterilized. For example, the sealing adhesive may be formulated to release upon exposure to an additional chemical reaction or energy, such as UV wavelength radiation. Alternatively, the sealing adhesive may be formulated to peel away from the substrate it bond to upon application of certain level of force or application of force in a particular direction.

Any combination of such functionalities and/or additional functionalities may be incorporated in the formulation of the sealing adhesive.

The sealing adhesive can be applied to the surface of the sterilization wrap system in any manner that would provide coverage and placement of the adhesive as desired. Such application can be accomplished by any method commonly understood in the art. For example, the sealing adhesive could be printed on the surface of the sterilization wrap system. Alternatively, the adhesive may be applied by spray, swirl, bead or slot coating methods. The sealing adhesive could also be provided in roll form, unwound, applied and attached to the sterilization wrap system. Such an adhesive may also be incorporated into the production of the sheet material itself. One skilled in the art would see that there are a multitude of options in providing the sealing adhesive to the surface of the sterilization wrap system.

The sealing adhesive 26 can be present on the sterilization wrap system 10 in various locations and configurations. The exact location and configuration can be designed to best address the method of wrapping. As shown in FIGS. 2 and 4, the sealing adhesive 26 can cover one entire surface of the sterilization wrap system 10. FIG. 1 through FIG. 4 show the sealing adhesive on the second exterior surface 46 of the sterilization wrap system, which is the surface facing opposite the surface that contacts the sterilization tray 18. Alternatively, the sealing adhesive 26 may be present on only the first exterior surface 44 or on both the first and second exterior surfaces 44, 46 of the sterilization wrap system 10 (not shown).

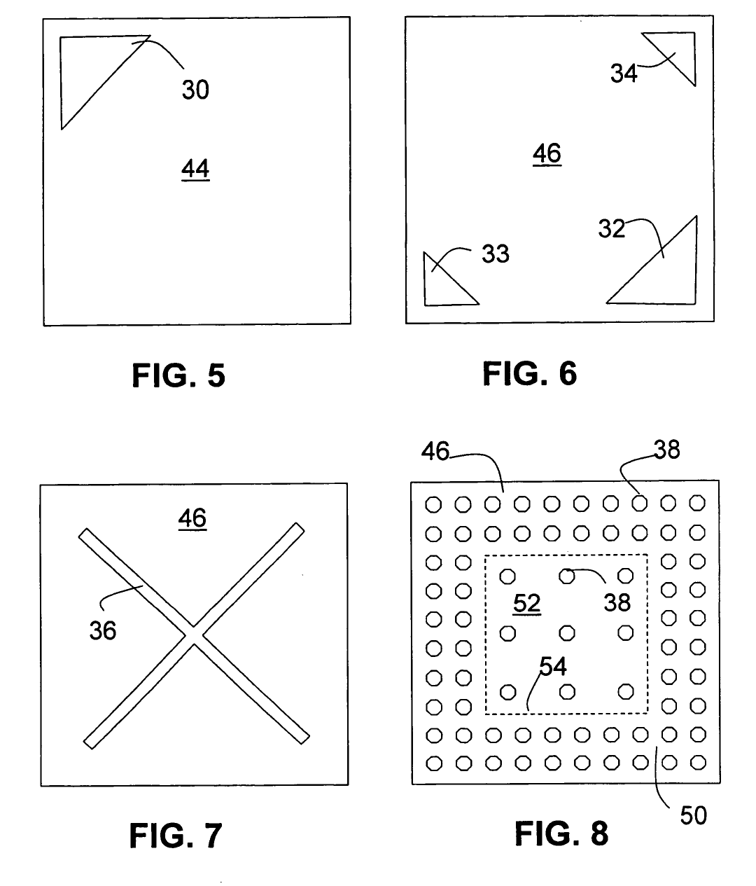

Another embodiment of the invention is the placement of the sealing adhesive 26 on the surface of the sterilization wrap system in discrete areas such that the total area covered by adhesive is less than the total surface area of the sterilization wrap system. Such discrete adhesive patterns could be present on either the first exterior surface 44 or the second exterior surface 46, or on both the first and second exterior surfaces 44, 46. For example, FIG. 5 illustrates the sealing adhesive placed in a single adhesive area 30, namely the corner of the first exterior surface 44. The single adhesive area 30 is shown as triangular in shape for illustrative purposes, but it could be of any shape or size as required for the particular needs of the sterilization wrap system.

The sealing adhesive could also be placed in multiple discrete areas. In the example of FIG. 6, sealing adhesive is present on the second exterior surface in three discrete locations. The sealing adhesive is shown present in first corner adhesive area 32, as well as in a second corner adhesive area 33 and a third corner adhesive area 34. The size of the first corner adhesive area 32 can be larger than both the second and third corner adhesive areas 33,34 (as shown), all three of the areas could be of the same size, or each of the three could be different sizes. All three of the corner adhesive areas are also shown as being triangular in shape, but they can be any shape. The total adhesive area in this example would be the sum of the surface areas of the individual adhesive areas and would be less than the total surface area of the sterilization wrap system.

As shown in FIG. 7, another exemplary embodiment of discrete adhesive placement would be a crisscross adhesive pattern 36. The elements of the crisscross pattern could alternatively extend all the way to the corners or may be shorter than shown. The pattern could also be wider or narrower than shown. Other shapes and patterns are also envisioned as possible arrangements of the sealing adhesive. Rather than the crisscross pattern, as seen in FIG. 7, the adhesive could be located on either the first or second exterior surfaces 44,46 (or both surfaces) in a pattern of parallel rows, a cross-hatch pattern, a spiral pattern, a parallel sinusoidal pattern, or other similar patterns.

In another embodiment, the sealable adhesive 26 may be placed on the sterilization wrap system 10 in a plurality of localized, discontinuous adhesive points 38, such as are shown in FIG. 8. These adhesive points may be uniformly spaced across the surface of the sterilization wrap system 10 or they may be broken into two or more zones with each of these zones having varying degrees or densities of adhesive points. Referring specifically to FIG. 8, the sterilization wrap system 10 is divided into a first zone 50 and a second zone 52 which, for purposes of illustration, are shown in FIG. 8 as being separated by an imaginary dashed line 54. The first zone 50 has a greater number of the overall plurality of adhesive points per unit area than the second area 52. In addition, the first zone 50 completely surrounds the second zone 52 thereby creating a sterilization wrap system 10 wherein the periphery of the sterilization wrap system 10 has a generally greater degree of adhesive points than the central portion of the sterilization wrap system 10.

Additional patterns are of various sizes and shapes are contemplated. The pattern can be present on either the first exterior surface 44 or the second exterior surface 46, or can present on both such surfaces. If the sealing adhesive is present on both the first and second exterior surfaces 44, 46, the pattern may be the same on both surfaces or may be a different pattern on each surface. The size, location and shape of the pattern could be different on the first exterior surface 44 than is present on the second exterior surface 46. The total adhesive area could also be the same on both the first and second exterior surfaces 44, 46 or can be different. The placement, total area of adhesive and pattern can be selected to fit the needs of the particular sterilization wrap system or method of folding the same.

The design of the sealing adhesive 26 can also be used a visual cue for the end user to differentiate the first exterior surface 44 from the second exterior surface 46. For example, the presence of sealing adhesive 26 on only one surface, or the having two different patterns on the opposing surfaces, can help the end user determine which surface is which and help them align and manipulate the sterilization wrap system properly.

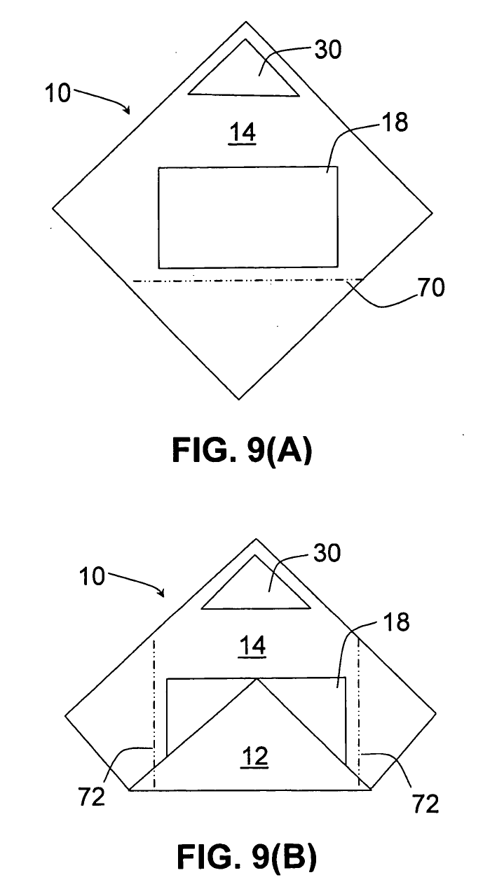

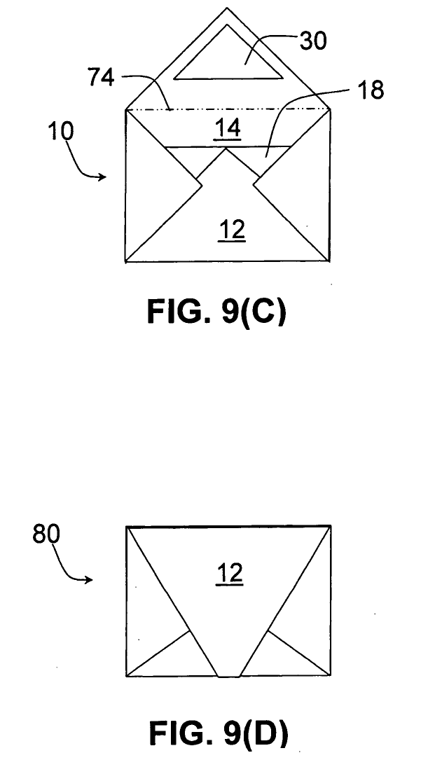

The placement, size, and design of the sealing adhesive 26 as shown and described above are only a few illustrative examples. One skilled in the art would see that other placement, sizes of adhesive areas and designs are possible and can be created to cooperate with the sterilization wrap system and folding procedure utilized. One such folding procedure is shown sequentially in the FIG. 9(A) through FIG. 9(D). These figures show the folding of a multiple ply, single step sterilization wrap system 10, such as illustrated in FIG. 3, about an item to be sterilized by a simultaneous wrapping procedure. The item to be wrapped in this example is a sterilization tray 18. While a multiple ply, single step sterilization wrap system 10 is illustrated in FIG. 9(A)-9(D) and is discussed in this example, a similar such procedure could be preformed using a single ply sterilization system, such as illustrated in FIG. 1, made according to the present invention.

The wrapping procedure begins by placing the sterilization tray 18 on top of the sterilization wrap system. As shown in FIG. 9(A), the sterilization wrap system 10 used for this example has a single adhesive area 30, such as shown in FIG. 5, on the surface of the second sheet 14. The sterilization tray 18 is place in the center of the sterilization wrap system 10, which is oriented such that the single adhesive area 30 is in the top corner. The bottom corner of the sterilization wrap system is folded along the first imaginary fold line 70 such that the folded bottom corner rests on top of, and partially covers, the sterilization tray 18. As can be seen in FIG. 9(B), once the bottom corner is folded over, the first sheet 12 will be exposed while the second sheet 14 will be in contact with sterilization tray 18.

The second step is to fold the left and right corners of the sterilization wrap system 10 along the second imaginary fold lines 72, as shown in FIG. 9(B). The folded left and right corners cover more of the sterilization tray 18, as shown in FIG. 9(C). The final step is to fold the upper corner, having the single adhesive area 30, along the third imaginary fold line 74. As shown in FIG. 9(D), this last fold completely encloses the sterilization tray 18 within the folds of the sterilization wrap system 10 to form a wrapped package 80. With this last fold, the upper corner is large enough such that when folded over it can be tucked on the other side of the wrapped package 80.

The entire surface of the wrapped package 80 will be the surface of the first sheet 12, while the enclosed sterilization tray 18 will only be in contact with the surface of the second sheet 14. Also, with last fold the single adhesive area 30 of the upper corner of the sterilization wrap system 10 will be in contact with the portion of first sheet 12 that was exposed when the first fold of the bottom corner was made.

The package is then transferred to the sterilizing apparatus where it is exposed to sterilizing conditions. The sealing adhesive of the single adhesive area 30 is activated upon this exposure to the sterilizing conditions and bonds to the surface of the first sheet 12 in the area that it contacts the first sheet 12.

According to other embodiments of the present invention, the sealing adhesive is adapted to avoid undesirable sticking to other portions of the sterilization wrap system, to other sterilization wrap systems prior to use, to the items to be sterilized, or to other items, prior to activation upon exposure to the sterilizing conditions. Such undesirable adhesion would cause such sterilization wrap systems to become cumbersome and frustrating to use. One possible desired sealing adhesive would be formulated such that it would only become sticky upon exposure to the sterilization conditions. For example, such a sealing adhesive would be a non-tacky coating before exposure to sterilization conditions. Alternatively, the sealing adhesive may have some tack when applied to the surface of the sterilization wrap system, but would rendered non-tacky by a coating until exposed to sterilization conditions.

Another possible sealing adhesive that avoids undesirable adhesion would be the use of a sealing adhesive that was tacky prior to activation, along with a release material. Such a release material could be made of paper, plastic, a nonwoven fabric, or the like. The release material would be removed upon the final folds of the sterilization wrap to expose the sealing adhesive and allow the tacky sealing adhesive to hold the final fold of the sterilization wrap system prior to exposure to the sterilization conditions. The release material can be designed to be removed in its entirety, or could be designed such that it could be selectively removed only in particular regions that the user wishes to expose the sealing adhesive.

This would be similar to the adhesive with release paper found commonly on shipping envelopes. Such envelopes have a pressure sensitive adhesive printed on the closing flap that is covered by a silicone-coated release paper. The release paper is removed to expose the adhesive. The flap is then folded over on to the body of the envelope and pressed to seal the envelope closed.

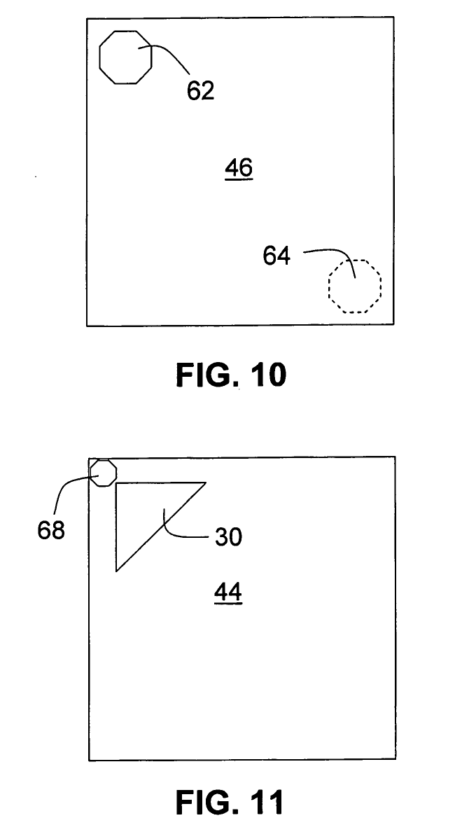

It may also be possible to formulate a sealing adhesive that only adheres to a specific receptive surface coating. This receptive surface coating may be another surface coated with the same sealing adhesive or some other selective coating. One possible embodiment of this type of selective sealing adhesive on a sterilization wrap system 10 is shown in FIG. 10. In FIG. 10, the sealing adhesive is located a selective adhesive area 62 on the second exterior surface 46 of the sterilization wrap system 10. The receptive surface area 64 is located in the opposite corner, and opposite side, of the sterilization wrap system 10 from the selective adhesive area 62. Upon folding the sterilization wrap system 10 of FIG. 10, as previously discussed for FIG. 9(A)-9(D), the selective adhesive area 62 would end up proximate to the receptive surface area 64 such that the two areas adhere to one another.

Another embodiment of the present invention is the incorporation of a secondary fastening means to hold the folds of the sterilization wrap system in place until it is exposed to sterilization conditions and the sealing adhesive is activated and holds the sterilization wrap system closed. Such a secondary fastening means would only need to hold the sterilization wrap system closed during the time the wrapped package is formed until the wrapped package is moved to the sterilization equipment. Alternatively, it is possible that the secondary fastening means may be strong enough to hold the wrapped package closed by itself, wherein the sealing adhesive would provide added closure strength as well as providing a barrier. The secondary fastening means can be a permanent part of the sterilization wrap system or can be designed to be removed after the wrapped package is exposed to the sterilization conditions.

A secondary fastening means can be located anywhere on the sterilization wrap system that will allow the secondary fastening means to hold the sterilization wrap in a closed configuration until it is exposed to sterilization conditions. For example, as shown in FIG. 11, the sterilization wrap system having a single adhesive area 30, such as shown in FIG. 5, could be adapted to include a secondary fastening means 68. One skilled in the art can clearly understand that the secondary fastening means 68 could be bigger or smaller, of a different shape, or in a different position than shown in the exemplary illustration of FIG. 5.

One possible method of providing secondary fastening means are means alluded to earlier. The sealing adhesive may have certain level of tackiness such that the adhesive could help hold the wrapped package closed, but not so tacky that the sterilization wrap system would be difficult to use. Also discussed was the possibility of using a tacky sealing adhesive along with a release material that could be removed from the sealing adhesive just prior to closing the wrapped package. It is also possible that a secondary fastening adhesive could be used in a discrete area to merely hold the final folds of the sterilization wrap system closed. Finally, one could continue the current practice of using an adhesive tape to hold the final fold of the sterilization wrap system closed. With the use of the sealing adhesive, such an adhesive tape could be less aggressive than currently used and/or could be removed post-sterilization, but prior to package entering the sterile environment.

Another possible secondary fastening means could be the use of passive fastening means. The tuck of the final corner of the wrapped package can be all that is required to hold the package closed until exposed to sterilization conditions. Similarly, the weight of the item to be sterilized within the wrapped package could be used to hold the tip of the final corner closed beneath the item by the mere weight of the item. Alternatively, the tip of the final corner can be designed to include a weight such that the final corner would be held shut by the weighing down of the corner. Other such passive fastening means may include the addition of stiffened zones or regions on the wrap corresponding that are able to hold a fold or crease. Such stiffened areas may be provided by a coating added to the wrap material, additional bonding, the addition of a wire mesh, or some other method that would allow for a more tenacious fold or crease.

A secondary fastening means can include the use of magnetic attraction to keep the wrapped package in a closed configuration until exposure to sterilization conditions. Magnets could be incorporated into the sterilization wrap system such that when wrapped in a specific manner the magnets would hold the wrapped package closed by their attraction to one another or to the enclosed metal sterilization tray. Similarly, a static charge could be imparted to the sterilization wrap system, or a surface applied to a portion of the sterilization wrap system, such that static attraction would hold the wrapped package closed about the item to be sterilized up until its exposure to sterilization conditions.

In yet another embodiment the secondary fastening means can be mechanical fastening means. Such mechanical fastening means can also be provided by interlocking geometric shaped materials, such as hook, loops, bulbs, mushrooms, arrowheads, balls on stems, male and female mating components, clips, buckles, snaps, buttons, or the like. In particular embodiments, the fastening components and mating fastening components comprise hook-and-loop fastening elements. One skilled in the art will recognize that the shape, density and polymer composition of the hooks and loops may be selected to obtain the desired level of fastening between the fastening components and the mating fastening components.

Of course, the present invention encompasses fastening means in which one or more mechanical fasting systems and adhesives may be used separately or together. The presence of both an adhesive strip and mechanical fastening means in a sterilization wrap system gives added options in terms of how the sterilization wrap system can be held securely closed until exposure to sterilization conditions.

We claim:

1. A sterilization wrap system comprising:
at least a first sheet having a periphery, an upper surface and a lower surface, where the lower surface has a total area; and
an adhesive on the lower surface of the first sheet, where the adhesive has a total adhesive area and where the adhesive is activated by sterilization conditions.

2. The system of claim 1, further comprising a second sheet having a periphery, an upper surface and a lower surface, where the first and second sheets are joined together at their peripheries, with the lower surface of the second sheet facing the upper surface of the first sheet.

3. The system of claims 1, where the first and second sheets are spunbond/meltblown/spunbond nonwoven web laminates.

4. The system of claim 3, where the first and second sheets contain polypropylene.

5. The system of claim 1, where the adhesive is activated by steam sterilization conditions.

6. The system of claim 1, where the adhesive is activated by ethylene oxide sterilization conditions.

7. The system of claim 1, where the adhesive is activated by hydrogen peroxide plasma sterilization conditions.

8. The system of claim 1, where the adhesive is placed in at least one discrete zone of the lower surface of the second sheet and where the total adhesive area is less than the surface area of the lower surface.

9. The system of claim 1, where the adhesive indicates if it has been activated.

10. The system of claim 9, where the adhesive indicates that it has been activated by changing colors when activated.

11. The system of claim 1, where the system further comprises a secondary fastening means.

12. A wrapped package formed by the combination of a sterilization wrap system and an article to be sterilized comprising:
an article to be sterilized; and
a sterilization wrap system comprising a first sheet, a second sheet, and an adhesive present on at least a portion of the first sheet; the first sheet comprising a spunbond/meltblown/spunbond nonwoven web laminate; the second sheet comprising a spunbond/meltblown/spunbond nonwoven web laminate;
where the article to be sterilized is positioned on the sterilization wrap system and is wrapped by the folding of the sterilization wrap system about the article; and
where the adhesive is activated by sterilization conditions to form a bond which holds the sterilization wrap system in a closed configuration to form the wrapped package.

13. The wrapped package of claim 12, where the article to be sterilized is at least one reusable medical instrument.

14. The wrapped package of claim 12, where the first and second sheets are joined together.

15. The wrapped package of claim 12, further comprising a secondary fastening means which also holds the sterilization wrap system in a closed configuration.

16. A method of sterilizing an article comprising:
providing an article;
wrapping the article with a sterilization wrap system, where the sterilization wrap system comprises (a) a first sheet comprising a spunbond/meltblown/spunbond nonwoven web laminate, (b) a second sheet comprising a spunbond/meltblown/spunbond nonwoven web laminate and (c) an adhesive applied to the first sheet, where the adhesive is activated under sterilization conditions; and
exposing the wrapped article to sterilization conditions for a sufficient time to substantially sterilize the article.

17. The method of claim 16, where the sterilization conditions are selected from steam sterilization conditions, ethylene oxide sterilization conditions, and hydrogen peroxide plasma sterilization conditions.

18. The method of claim 16, further comprising the step of securing the sterilization wrap system in a closed configuration with a secondary fastening means after wrapping the article and prior to exposing the wrapped article to sterilization conditions.
